(19) United States
(12) Patent Application Publication
(10) Pub. No.: US 2016/0282297 A1
(43) Pub. Date: Sep. 29, 2016

(54) Gas Sensor For Detecting Nitrogen Oxides, And Operating Method For Such A Gas Sensor

(71) Applicant: SIEMENS AKTIENGESELLSCHAFT, München (DE)

(72) Inventors: Sabine Fischer, Stuttgart (DE); Maximilian Fleischer, Hoehenkirchen (DE); Erhard Magori, Feldkirchen (DE); Ralf Moos, Bayreuth (DE); Roland Pohle, Herdweg (DE)

(73) Assignee: Siemens Aktiengesellschaft, Muenchen (DE)

(21) Appl. No.: 15/033,790

(22) PCT Filed: Oct. 23, 2014
(86) PCT No.: PCT/EP2014/072712

§ 371 Date: May 2, 2016

(30) Foreign Application Priority Data
Oct. 31, 2013 (DE) 102013222195.9

(51) Int. Cl. G01N 27/407; G01N 33/00; G01N 27/30

(52) U.S. Cl. CPC G01N 27/4071; G01N 27/4074; G01N 27/30; G01N 33/0037

(57) ABSTRACT

A gas sensor for detecting nitrogen oxides in a gaseous mixture, including an oxygen ion conductor and at least two electrodes mounted on the oxygen ion conductor, the electrodes consisting of the same material, and wherein the gas sensor is designed so that during operation of the gas sensor both electrodes come into contact with the gaseous mixture.

### CROSS-REFERENCE TO RELATED APPLICATIONS

This application is a U.S. National Stage Application of International Application No. PCT/EP2014/072712 filed Oct. 23, 2014, which designates the United States of America, and claims priority to DE Application No. 10 2013 222 195.9 filed Oct. 31, 2013, the contents of which are hereby incorporated by reference in their entirety.

### TECHNICAL FIELD

Rising requirements with respect to the emission of waste gases and with respect to efficiency in the operation of power stations, heating systems, garbage-incineration plants, gas turbines and engines of all types can be met, inter alia, by the composition of gases in the respective plants and systems being determined in ongoing operation and being evaluated for improved operation. This results in a need for sensors for determining components of a gas mixture.

### BACKGROUND

An example of this is the steadily increasing number of motor vehicles, for which at the same time ever more stringent exhaust-gas regulations are to be complied with, in order to limit the damage to the environment and health that is caused by exhaust gases of combustion. Of the harmful components of exhaust gas, besides sulfur oxides and carbon dioxide the group of the nitrogen oxides, called NOx for short, is moving more and more into the foreground. In order to diminish the emissions of nitrogen oxides, enormous efforts are being made both technically and financially: for example, exhaust-gas recirculation and selective catalytic reduction (SCR). For the purpose of monitoring the functioning of these processes and for the purpose of lowering the operating costs, an ongoing monitoring of the NOx concentration in the exhaust gas of the vehicle is necessary.

Especially in automotive applications, in certain countries it is prescribed that the operational capability of the exhaust-gas after treatment system be diagnosed in the vehicle itself. The automobile manufacturer has to ensure that a randomly selected vehicle complies with the emission regulations, even after a long period of running. Above all for diesel vehicles, the monitoring of NOx storage catalysts and SCR catalysts for the purpose of diminishing the NOx emissions is a task on which work is proceeding intensively.

Besides arising as exhaust gases of combustion, nitrogen oxides may also arise as process gases in chemical plants. Here too the detection of the nitrogen oxides may be of interest.

Known sensors for the measurement of NOx are optical or chemiluminescence-based systems. Besides their high price, these systems have the disadvantage that an extractive measurement is necessary, i.e. a withdrawal of gas is needed. For many applications this is associated with high costs.

Known sensors that overcome these disadvantages are based on yttrium-stabilized zirconia (YSZ); in this case, electrodes of the same material, for example consisting of platinum, come into operation. The operating principle in this case is based on a two-chamber system with simultaneous measurement of oxygen and NOx. But a complex structure and therefore high price is still a disadvantage here.

In contrast, so-called mixed-potential sensors are also known which include electrodes consisting of different materials and which evaluate as sensor signal the potential difference between these electrodes.

From US 2005/0284772 A1 a measuring method is known in which use is made of zirconia-based lambda probes or mixed-potential sensors in order to construct an NOx sensor. A dynamic method serves as measuring principle in this case, wherein defined voltage pulses are applied to the sensor, and the respective gas-dependent depolarization is measured. The discharge curves recorded in this way exhibit a strong dependence on the surrounding gas atmosphere. In this case, nitrogen oxides can be distinguished well from other gases.

One principle of the lambda probe is that one of the electrodes faces towards the gas mixture to be gauged, whereas the other electrode faces towards a gas having a defined partial pressure of oxygen. The sensors that are used as such—that is to say, the lambda probes—continue to exhibit the known disadvantages listed at the outset.

### SUMMARY

One embodiment provides a gas sensor for detecting nitrogen oxides in a gas mixture, including an oxygen-ion conductor and at least two electrodes arranged on the oxygen-ion conductor, the electrodes consisting of the same material, wherein the gas sensor has been designed in such a manner that in the course of operation of the gas sensor both electrodes come into contact with the gas mixture.

In a further embodiment, the gas sensor includes a heating device configured for heating the oxygen-ion conductor and the electrodes to a temperature at which a conduction of oxygen ions is present.

In a further embodiment, the gas sensor includes three or four electrodes, the electrodes consisting of the same material and having been arranged in such a manner that in the course of operation of the gas sensor they come into contact with the gas mixture.

In a further embodiment, the oxygen-ion conductor is porous.

In a further embodiment, the electrodes have been configured as interdigital electrodes.

In a further embodiment, all the electrodes come into contact with the gas mixture.

Another embodiment provides an operating method for a gas sensor for detecting nitrogen oxides in a gas mixture, wherein use is made of a gas sensor which comprises an oxygen-ion conductor and at least two electrodes arranged on said conductor, the electrodes consisting of the same material, and the gas sensor is associated with the gas mixture in such a manner that both electrodes come into contact with the gas mixture.

In a further embodiment, the oxygen-ion conductor and the electrodes are maintained at a temperature of at least 350° C.

In a further embodiment, use is made of a gas sensor with three or more electrodes, and a phase-shifted polarization and readout of the mutual potentials is carried out.

In a further embodiment, for the purpose of generating a signal, alternately a voltage is applied between the electrodes, or a flow of current through the electrodes is generated and the voltage progression is measured.

In a further embodiment, the polarity of the applied voltage alternates.

In a further embodiment, the phase in which the voltage progression is measured is concluded after reaching a termination criterion, in particular after expiration of a definable period of time or upon reaching a definable voltage.

In a further embodiment, the polarization current in the case of polarization by means of a voltage, or the polarization voltage in the case of polarization by means of a defined current, and/or the depolarization voltage with defined depolarization time, or the depolarization duration with defined depolarization voltage serves as sensor signal.

### BRIEF DESCRIPTION OF THE DRAWINGS

Example aspects and embodiments of the invention are described below with reference to the figures, in which:

FIG. 1 shows a first variant of a gas sensor with two electrodes, according to one embodiment,

FIG. 2 shows a diagram for the measuring method for operating the gas sensor, according to one embodiment,

FIG. 3 shows a second variant of a gas sensor with three electrodes, according to one embodiment, and

FIG. 4 shows a third variant of a gas sensor with a heating device, according to one embodiment.

### DETAILED DESCRIPTION

Embodiments of the present invention provide a gas sensor and an operating method for a gas sensor having a simplified structure.

The disclosed gas sensor for detecting nitrogen oxides in a gas mixture may comprise an oxygen-ion-conducting material and at least two electrodes arranged on the ion-conducting material, the electrodes consisting of the same material. The gas sensor has been designed in such a manner that both electrodes come into contact with the gas mixture in the course of operation of the gas sensor.

For the invention it was recognized experimentally that for the detection and determination of the concentration of nitrogen oxides it is not necessary that one of the electrodes—with the same electrode material—is in contact with a fixed partial pressure of oxygen—that is to say, for example, with the ambient air. Rather, it was surprisingly discovered that a detection of nitrogen oxides is possible if two electrodes of the same material are both in direct contact with the gas mixture to be gauged. This contradicts the opinion, previously advocated in the state of the art, relating to the operation of this type of sensor.

As a result, it surprisingly becomes possible to simplify the structure of the NOx gas sensor considerably. Accordingly, on the one hand it is possible to manufacture the electrodes from the same material, saving several elaborate steps in the course of production. But, at the same time, it is no longer necessary to design the structure in such a way that one of the electrodes is in contact with a reference gas and has been isolated from the gas mixture to be gauged. Since the reference gas is customarily the ambient air, in the state of the art an entrance, for example, for the ambient air to an inside, formed as a chamber, is created for this in the zirconia, requiring a considerable effort in production. Consequently, besides the more favorable production, a saving can also be made on expensive raw materials, for example by means of planar technology. Moreover, the sensor has a far better potential to be made very small.

The disclosed gas sensor, on the other hand, may be of comparatively simple construction, since both electrodes have been manufactured from the same material and both electrodes merely have to come into direct contact with the gas mixture.

The gas sensor expediently includes electrical connections to the electrodes and means for applying a voltage to said electrodes, as well as a device for measuring the voltage between the electrodes during the subsequent depolarization.

The ion-conducting material may be, for example, yttrium-stabilized zirconia (YSZ). Said material may even act as carrier for the electrodes. Alternatively, it is also possible that the ion-conducting material has been applied as a layer on a carrier, for example consisting of alumina. The electrodes, in turn, have then expediently been applied on the layer of the ion-conducting material. The electrodes themselves are expediently made of platinum.

It may be advantageous if the gas sensor includes a heating device that has been configured to heat the sensor, in particular the ion-conducting material and the electrodes, to a temperature at which a conduction of oxygen ions is present. It has been discovered experimentally that the measurement of nitrogen oxides works best starting from this operating temperature. The heating device may, for example, have been configured as an electric heater in the form of a flat layer of platinum, for example. Said device has expediently been electrically isolated from ion-conducting material and, of course, from the electrodes by a layer of insulator, for example by the carrier.

In one embodiment the ion-conducting material may have been realized as porous material. In the case of a sensor from the state of the art, in which the ion-conducting material borders both the gas mixture to be gauged and, for example, ambient air, the gradients in the partial pressure of the various gases lead to a diffusion of the gases through the ion-conducting material, resulting in a deterioration of the sensor signal. Since in the case of the present sensor the ion-conducting material no longer adjoins the ambient air but is expediently surrounded on all sides by the gas to be gauged, no such diffusion happens any longer, and use may be made of a porous, in particular open-pored, material. A porous ion-conducting material can advantageously be produced more easily, is more stable in relation to the loads due to fluctuating temperatures, and exhibits a higher specific surface area, affording advantages for the interaction with gases, and therefore for the sensor signal.

For the purpose of gauging, a voltage may be applied to the pair of electrodes for a definable first time-interval of between 0.1 s and 1 s, e.g., 0.5 s. Thereafter the discharge is observed for a second time-interval, and the voltage is recorded. The voltage level after a time-interval of 3 s, for example, is then the sensor signal. This procedure is then repeated. It is very advantageous in this case if the polarity of the voltage applied in the first time-interval is alternately reversed.

According to one embodiment, the gas sensor includes three or four electrodes. In this case, two of the electrodes, for example, may have been arranged on one side of the ion-conducting material, whereas the third electrode or the third and fourth electrodes has/have been arranged on the other side of the ion-conducting material. With the additional electrodes it is possible for several improvements to be obtained. Accordingly, the impressing of a voltage during a respective first time-interval for the various pairs of electrodes may be undertaken with temporal offset—in other words, shifted in phase. Hence a point of measurement is generated more frequently, and hence the temporal resolution is improved. Alternatively or additionally, pairs of electrodes may be connected in series, and hence an improvement of the signal deviation may be obtained.

The electrodes may be geometrically designed in order to obtain an improvement of the signal quality. For example, the electrodes may be designed as finger electrodes (interdigital electrodes).

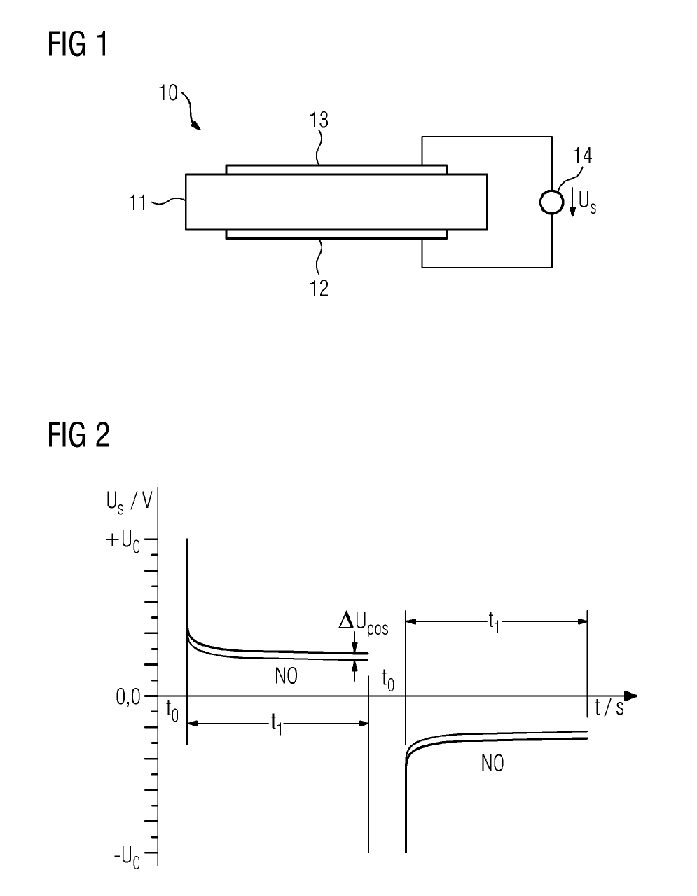

FIG. 1 shows, in greatly schematized form, a first gas sensor 10 according to one embodiment. Said sensor comprises a block 11 of YSZ material. On a first side of this block 11 a first platinum electrode 12 has been arranged, whereas on a second side, which is situated opposite the first side, a second platinum electrode 13 has been applied. The platinum electrodes 12, 13 have been electrically connected to a device 14 for generating and measuring voltage Uₛ. Not represented in FIG. 1 are means by which the first gas sensor 10 can be introduced into a space filled with the gas mixture to be gauged, for example a flange to be screwed into a correspondingly configured opening. These means and the gas sensor 10 have been designed in such a way that after mounting of the gas sensor 10 both the first and the second platinum electrode 12, 13 are directly in contact with the gas mixture. On the other hand, a contact of the block 11 with the ambient air, for example, is expediently avoided in this case.

In operation of the gas sensor 10 a voltage Uₛ is applied alternately between the platinum electrodes 12, 13 by means of the device 14, and the voltage progression is gauged. An exemplary progression of the voltage Uₛ is represented in FIG. 2.

Accordingly, a fixed, positive voltage is applied from left to right in FIG. 2 during a first time-interval t₀. The voltage used here may amount to between 0.5 V and 2 V. The duration of the first time-interval t₀ may amount to between 0.1 s and 1 s. During the ensuing second time-interval t₁ the voltage Uₛ drops (numerically), the progression being influenced by the presence of NOx in the gas mixture. In the following, a fixed voltage with negative polarity is applied during a further second time-interval t₀, and, following this, the progression of the voltage Uₛ is tracked in a further second time-interval. A measured value may be taken in this case, for example, after expiration of a fixed time within the second time-interval t₁, for example after 1 s or 3 s.

Surprisingly, it becomes evident experimentally that a usable NOx signal can be measured during the first time-interval t₀ for both polarities of the applied voltage. In the case of a sensor that utilizes an air reference—that is to say, in which an electrode has been exposed to the ambient air instead of the gas mixture-only a very weak signal is generated in the case of one of the polarities. This results in an improved frequency of measurement, since a signal is available twice as frequently.

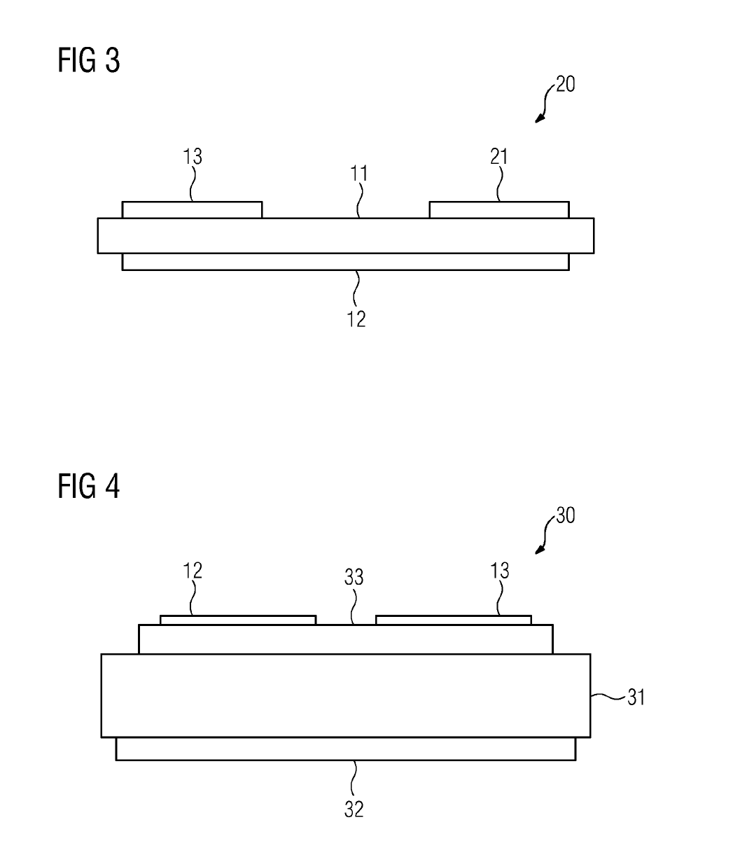

FIG. 3 shows, likewise in greatly schematized form, a second gas sensor 20 according to one embodiment, which has been constructed in a manner similar to the first gas sensor 10 and is operated in a manner similar to that for the first gas sensor 10. Said sensor comprises a block 11 of YSZ material. On a first side of this block 11 a first platinum electrode 12 has been arranged, whereas on a second side, which is situated opposite the first side, a second platinum electrode 13 has been mounted. The platinum electrodes 12, 13 have, as in the case of the first gas sensor 10, been electrically connected to a device 14 for generating and measuring voltage Uₛ. In contrast to the first gas sensor 10, the second platinum electrode 13 is not exactly as large as the first platinum electrode 12 but exhibits a smaller surface. Besides the second platinum electrode 13, a third platinum electrode 21 has been provided, likewise on the second side of the block 11.

In the case of the second gas sensor 20, the device 14 for generating a voltage, which is no longer represented in FIG. 3, has been configured to be correspondingly more complex, so that different potentials between the electrodes 12, 13, 21 can be generated. In this way, a positive potential between the first and second electrodes 12, 13 can be generated in ongoing operation, for example in the first time-interval, whereas a negative potential is generated between the first and third electrodes 12, 21. Hence two independent measuring signals can be recorded in the course of the following second time-interval. Hence the signal accuracy, for example, can be improved.

If the respective first and second time-intervals—that is to say, also the instants at which the measuring signals are recorded—are drawn up with a temporal offset, the temporal resolution of the measuring signals is improved. This effect can also be intensified further with, for example, four or five electrodes if a corresponding phase shift is provided in the electrical drive. Given a sufficient quantity of electrodes, an interconnection of pairs of electrodes is also possible, in order to obtain an improved signal deviation.

FIG. 4 shows a third gas sensor 30 according to a further embodiment of one embodiment. The third gas sensor 30 has been constructed on an alumina substrate 31. On one side of the substrate 31 a layer 33 of zirconia has been applied, for example by screen printing. On this layer, in turn, the first and second platinum electrodes 12, 13 have been arranged side by side. On the rear of the substrate 31 a platinum heating structure 32 has been applied. This structure has been configured to be able to heat the third gas sensor to 350° C. For the purpose of temperature control, on the one hand use may be made of the heating structure 32 itself. Alternatively, it is also possible that an additional temperature detector has been provided for this. If the temperature of the gas mixture itself lies distinctly above 350° C., it may also be sufficient to operate the heating structure 32 only as a temperature detector, since an additional heating is unnecessary.

Besides a substrate 31 consisting of Al₂O₃, use may be made of other substrate materials, so long as they are expediently not ion-conducting. For the purpose of applying the layer of zirconia, as an alternative to screen printing use may also be made of an aerosol deposition, for example. In contrast to screen printing, this produces a dense layer.

1. A gas sensor for detecting nitrogen oxides in a gas mixture, with the gas sensor comprising:
an oxygen-ion conductor; and
at least two electrodes arranged on the oxygen-ion conductor, the electrodes being formed from the same material,
wherein the gas sensor is configured such that both electrodes come into contact with the gas mixture during operation of the gas sensor.

2. The gas sensor of claim 1, comprising heating device (32) configured to heat the oxygen-ion conductor and the electrodes to a temperature at which a conduction of oxygen ions occurs.

3. The gas sensor of claim 1, comprising three or four electrodes formed from the same material and arranged such that the three or four electrodes come into contact with the gas mixture during operation of the gas sensor.

4. The gas sensor of claim 1, wherein the oxygen-ion conductor is porous.

5. The gas sensor of claim 1, wherein the electrodes comprise interdigital electrodes.

6. The gas sensor of claim 1, wherein all electrodes of the sensor come into contact with the gas mixture during operation of the gas sensor.

7. A method for detect nitrogen oxides in a gas mixture, the method comprising:
providing a gas sensor including an oxygen-ion conductor and at least two electrodes arranged on said conductor, the electrodes being formed from the same material,
operating the gas sensor such that both electrodes come into contact with the gas mixture.

8. The method of claim 7, wherein the oxygen ion conductor and the electrodes are maintained at a temperature of at least 350° C.

9. The method of claim 7, wherein the gas sensor includes three or more electrodes, and the method comprises performing a phase-shifted polarization and readout of mutual potentials.

10. The method of claim 7, comprising generating a signal by a process including:
applying a voltage between the electrodes, or
generating a flow of current through the electrodes and measuring an associated voltage progression.

11. The method of claim 10, wherein the polarity of the applied voltage alternates.

12. The method of claim 7, comprising:
generating a flow of current through the electrodes and measuring an associated voltage progression during a measurement phase,
concluding the measurement phase upon expiration of a defined time period or upon reaching a defined voltage.

13. The method of claim 7, comprising generating a sensor signal comprising at least one of:
a polarization current,
a polarization voltage, a
depolarization voltage, or
a depolarization duration.
